(19) United States
(12) Patent Application Publication
(10) Pub. No.: US 2024/0173308 A1
(43) Pub. Date: May 30, 2024

(54) SYSTEM, METHOD, AND TREATMENT FOR THE OVERDOSE OF XYLAZINE CONTAINING SUBSTANCES

(71) Applicant: Griffin Katz, New York, NY (US)

(72) Inventor: Griffin Katz, New York, NY (US)

(21) Appl. No.: 18/427,797

(22) Filed: Jan. 30, 2024

Related U.S. Application Data
(60) Provisional application No. 63/428,780, filed on Nov. 30, 2022.

(51) Int. Cl. A61K 31/4409; A61K 9/00; A61K 9/12; A61K 31/52; A61K 31/5377; A61K 45/06

(52) U.S. Cl. CPC A61K 31/4409; A61K 9/0043; A61K 9/12; A61K 31/52; A61K 31/5377; A61K 45/06

(57) ABSTRACT

A system may include an introducer including a body having an inner volume, a delivery tip, and an actuator. A system may include medication within the inner volume of the body, the medication including at least one of a xylazine antidote and xylazine overdose support medications, wherein the introducer is configured to deliver at least a partial dose of the medication, when the actuator is manipulated, through the delivery tip and into a body cavity. A system may include an introducer including a body having an inner volume, a delivery tip, and an actuator. A system may include medication within the inner volume of the body, the medication including at least one of a xylazine antidote and xylazine overdose support medications, wherein the introducer is configured to deliver at least a partial dose of the medication, when the actuator is manipulated, through the delivery tip and into a body cavity.

### REFERENCE TO RELATED APPLICATIONS

This U.S. nonprovisional utility patent application claims priority to U.S. provisional patent application No. 63/428,780, entitled “A SYSTEM, METHOD, AND TREATMENT FOR THE OVERDOSE OF XYLAZINE-CONTAINING SUBSTANCES,” to Inventor Griffin Katz, filed on Nov. 30, 2022, the entirety of which is herein incorporated by reference.

### BACKGROUND

Xylazine is a non-opioid sedative and muscle relaxant, primarily utilized in veterinary medicine to sedate large animals such as horses, cattle, and deer. Furthermore, it is unknown if reversal agents for use only in veterinary medicine (eg, yohimbine hydrochloride, tolazoline hydrochloride) are safe or even effective in humans; therefore, these should never be used for humans

It belongs to a class of drugs known as alpha-2 adrenergic agonists. This means it acts on receptors in the central nervous system to produce sedative and analgesic effects—causing a rapid decrease in the release of norepinephrine and dopamine in the central nervous system. It is structurally similar to levamisole, a veterinary drug that at one time was approved for human use but is no longer; clonidine, used to treat hypertension in humans and, infrequently, also involved in overdoses; and tizanidine, used as a muscle relaxer in patients with multiple sclerosis or spinal cord injuries. It has a variable duration of action in humans (8 to 72 hours), depending on the dose, route of administration, and drugs with which it is mixed, 1,2 Currently, there is no approved reversal agent for xylazine in humans. For its intended purpose in the veterinary field, xylazine has proven invaluable, facilitating procedures that would be unsafe or impossible on awake, unrestrained animals. However, for human use, it has not been approved by health regulatory agencies like the U.S. Food and Drug Administration (FDA).

In recent years, a concerning trend has emerged in the world of illicit drugs: the adulteration of street drugs with xylazine. Xylazine comes in a liquid form for veterinary use, but it can be converted into a salt or dried into a powder, which can be mixed into other powders or pressed into pills for illicit use, Often combined with opioids such as cocaine, heroin or fentanyl, xylazine has been detected in increasing numbers of overdose cases. Its addition to street drugs appears to enhance the depressant effects of opioids, leading to a heightened risk of respiratory depression, a leading cause of opioid overdose deaths. As users often are unaware of the presence of xylazine in their drugs, they unknowingly put themselves at increased risk with every use.

The reasons for xylazine's emergence in street drugs can be varied, ranging from its relative ease of acquisition from veterinary sources to its ability to augment or extend the effects of other narcotics, potentially offering drug dealers a way to increase profit margins. In humans, when xylazine is used as an adulterant, observed effects will vary based on substance combinations. Common adverse effects for xylazine alone include bradycardia, premature ventricular contractions, brief hypertension related to al activation followed by hypotension and other negative hemodynamic effects. Other symptoms include ataxia, diminished reflexes, disorientation, slurred speech, respiratory depression, sedation, and urinary incontinence, all of which can last from 8 to up to 72 hours. However, its addition comes with significant health risks. Beyond respiratory depression, xylazine has been associated with skin and soft tissue infections in intravenous drug users. [add Xylazine causes profound tissue injury, not only at the site of injection but locations remote to the site of injection The skin sores and ulcers associated with the drug have earned it another nickname: tranq or zombie drug. Xylazine-associated wounds can run so deep that they expose tendon and bone, necessitating amputation and spurring the demand for wheelchairs and crutches. Prolonged use of xylazine-containing substances can lead to impaired wound healing. The use of a substance intended for large animals and not approved for human consumption clearly poses serious public health concerns.

The presence of xylazine in illicit drugs underscores the dangers of unregulated drug markets where users cannot be certain of the contents or purity of their drugs. This highlights the importance of harm reduction strategies, such as drug checking services, to provide users with more information about their substances and the potential risks involved. As the use of xylazine in street drugs continues to rise, public health officials, law enforcement, and community organizations must be vigilant and collaborative in their efforts to address this growing threat.

[From JAMA: Although xylazine's life-threatening effects in humans—including central nervous system and respiratory depression, significant slowing of the heart rate, and low blood pressure-resemble those caused by opioids, it isn't an opioid, so naloxone doesn't reverse its effects”. As its use steadily increases, there is no approved antidote for xylazine overdose in humans. There is no antidote approved for human use. Hemodialysis has been suggested as a possible treatment. However, limited evidence suggests this intervention would not be effective. At this time, there is no standardized screening for xylazine overdose. Treatment must primarily focus first on ventilation and establishing hemodynamic stability and supportive treatment. The primary use of this invention is to reverse the effects of individuals suspected of overdosing on xylazine. The benefits include, but are not limited to, the reversal of xylazine-associated overdoses in a manner that is beyond supportive therapy. With this evidence and the difficulty of distinguishing opioid overdose and xylazine use, the FDA issued an alert in November 2022 to be aware of possible xylazine use in fentanyl, heroin, and other illicit drug overdoses and that naloxone may not be effective. Furthermore, it is unknown if reversal agents for use only in veterinary medicine (eg, yohimbine hydrochloride, tolazoline hydrochloride) are safe or even effective in humans; therefore, these should never be used for humans

Accordingly, it may be advantageous to provide systems, methods, and treatments that may reverse the effects of xylazine-associated overdoses in humans in ways that are not only supportive. Further, it may be advantageous to provide systems, methods, and treatments which are easily deliverable to people who are experiencing xylazine-associated overdoses.

### SUMMARY

In some aspects, the techniques described herein relate to a system for treating xylazine introduced into an animal body, including: an introducer including a body having an inner volume, a delivery tip, and an actuator; and medication within the inner volume of the body, the medication including at least one of a xylazine antidote and xylazine overdose support medications; wherein the introducer is configured to deliver at least a partial dose of the medication, when the actuator is manipulated, through the delivery tip and into a body cavity.

In some aspects, the techniques described herein relate to a xylazine overdose treatment device, including: an intranasal delivery device, including, a body including a reservoir configured to hold medication, a force receiving input device, a reservoir pressurizing mechanism configured to pressurize the reservoir when the force receiving input device receives a force input, and an aerosol nozzle coupled to the reservoir and configured to create an aerosol spray when the reservoir is pressurized; and a medication, disposed in the reservoir, including at least one of a xylazine antidote and xylazine overdose support medications.

In some aspects, the techniques described herein relate to a method of treating a xylazine overdose, including: identifying that a person has a xylazine overdose; providing a nasal spray device having a medication reservoir containing at least one of a xylazine antidote and xylazine overdose support medications; and spraying, using the nasal spray device, the medication into a nostril of a person.

In addition to the foregoing, other system aspects are described in the claims, drawings, and text forming a part of the disclosure set forth herein. The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the disclosures set forth herein.

### BRIEF DESCRIPTION OF THE DRAWINGS

FIG. 1 is an illustrative embodiment of a hand holding an exemplary intranasal spray device.

FIG. 2 is an example illustration of a nose of a patient depicted receiving medication through a nostril into a nasal cavity.

FIG. 3 is an example illustration of an alternative delivery method for a xylazine antidote or for xylazine overdose support medications.

FIG. 4 is an example illustration of capsules representative of a powder containing pill, a gel cap, a liquicap or the like.

FIG. 5 is an example flowchart of a method for treating a xylazine overdose.

The use of the same symbols in different drawings typically indicates similar or identical items unless context dictates otherwise.

### DETAILED DESCRIPTION

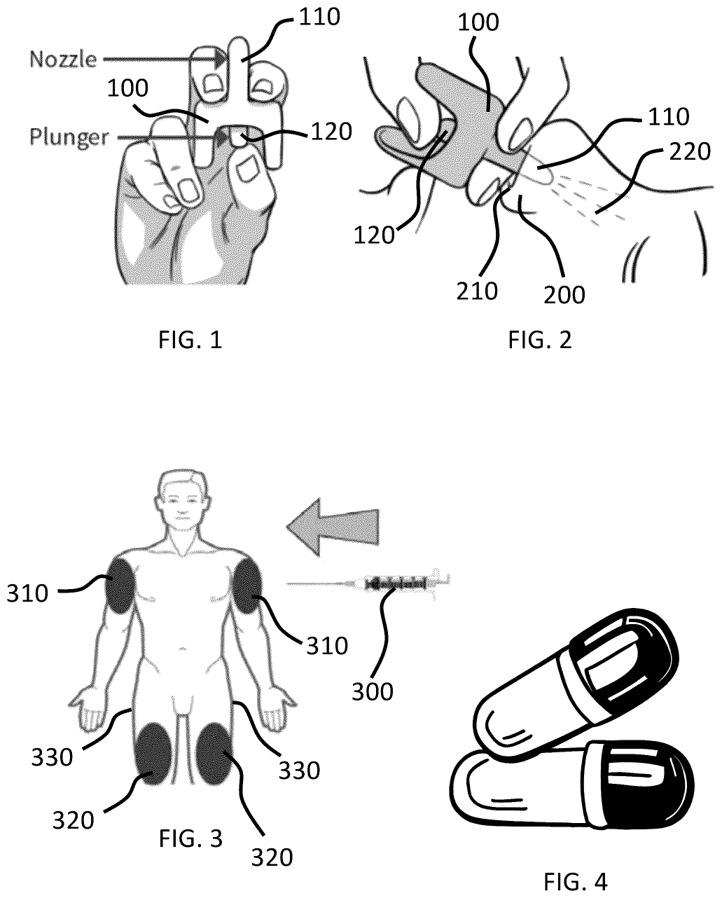

Referring to FIG. 1 In the medical field, it can be quite challenging to ascertain whether an individual has ingested xylazine or an opioid. This is primarily due to the fact that xylazine does not bind to the opioid receptor, a characteristic that differentiates it from opioids. As a result, naloxone, a medication used to block the effects of opioids, especially in overdose situations, will not have any effect on xylazine. Effects of xylazine usually manifest 15 to 30 minutes after administration, and sedation effects can last up to 4 hours. Once xylazine accesses to the vascular system, xylazine diffuses widely and through the blood-brain barrier decreasing norepinephrine and dopamine transmission in the central nervous system. Xylazine is metabolized by cytochrome P450 enzymes in the liver and excreted in urine. Because of serious adverse effects in humans, xylazine is not approved by the US Food and Drug Administration (FDA). Therefore, knowledge of xylazine's mechanisms of action in humans is significantly limited. Xylazine is approved only for veterinary use for dogs, cats, horses, elk, and deer.

If an individual presents with symptoms such as constricted pupils, shallow breathing, and bradycardia (a slower than normal heart rate), it may initially appear to be an opioid overdose. Xylazine use is also associated with disfiguring skin and soft tissue infections However, if the individual remains unresponsive even after multiple doses of naloxine, it may suggest the presence of xylazine instead. This is because naloxone should typically reverse the effects of an opioid overdose. What can family physicians do? Although evidence is limited regarding best practices, 15 family physicians should have an awareness of the drug supply and understand the potential implications of xylazine to provide comprehensive and holistic care. Treatment for xylazine intoxication is mainly supportive. Although other alpha-2 adrenergic agents (clonidine, tizanidine, guanfacine [Intuniv]) can assist with withdrawal, 17 the doses needed can exacerbate adverse effects of xylazine, such as bradycardia and hypotension. Benzodiazepines may be more effective for these individuals. Although clinician hesitancy to prescribe benzodiazepines is understandable, concerns can be allayed within a controlled acute care setting. Benzodiazepines may be appropriate in ambulatory settings, as well as selective serotonin reuptake inhibitors and other anxiety medications. Treatment of wound infections and wound debridement are important. Healing typically occurs over weeks to months, with the most effective treatment component being removal of the causative source (xylazine) whenever possible.

In such cases, xylazine should be suspected as the potential cause of the overdose. Xylazine is typically dried into a powder to facilitate its addition to opioids, making it difficult to identify by its physical properties and increasing the risk of a fatal overdose. Xylazine can exacerbate typical opioid adverse effects (e.g., bradypnea, apnea, bradycardia, hypotension) and, with repeated use, can lead to xylazine dependence and associated withdrawal symptoms (e.g., irritability, anxiety, dysphoria). However, to make a definitive diagnosis, further tests are required. One such test is a toxicology screen, which can detect the presence of various substances in the body. Specifically, gas chromatography-mass spectrometry (GC-MS) can be used. GC-MS is a method that combines the features of gas-liquid chromatography and mass spectrometry to identify different substances within a test sample. This test can confirm xylazine exposure by detecting its presence in the body.

Therefore, while clinical symptoms can provide initial clues, definitive diagnosis of xylazine use requires laboratory confirmation. It's important for healthcare providers to consider this possibility when treating patients with suspected drug overdoses, especially when they do not respond to standard treatments like naloxone. Because of the increasing likelihood that xylazine is involved, patients treated with naloxone need to be monitored for longer than before tranq came on the scene, Mason said. That's because naloxone could revive them but, if xylazine is present, they could slip back into unconsciousness

The prevalence of xylazine, a non-opioid sedative used in veterinary medicine, in overdose deaths has seen a significant increase in recent years. In 2015, xylazine was present in a mere 0.36% of overdose deaths. However, by 2020, this figure had risen dramatically to 6.7%, indicating an alarming trend.

The year-over-year increase from 2019 to 2020 was particularly notable, with the prevalence of xylazine in overdose deaths jumping by 44.8%. This suggests that the use of this substance is not only becoming more widespread, but that its rate of spread is accelerating.

In 2020, certain regions reported particularly high rates of xylazine presence in overdose deaths. In Philadelphia, for instance, xylazine was found in 31% of such cases. Maryland and Connecticut followed with rates of 19.3% and 10.2% respectively. From JAMA: In 2020, about 26% of overdose deaths in Philadelphia involved xylazine, the highest prevalence in the US, researchers reported in 2022. Maryland had the second-highest prevalence, with 19%, followed by Connecticut with 10%.

From JAMA: More recently, researchers found that a high percentage of paraphernalia samples collected at 8 Maryland syringe services programs between November 2021 and August 2022 tested positive for xylazine. Of 496 samples, 367 tested positive for an opioid—fentanyl or fentanyl analogues in all but 3 cases—and approximately 4 out of 5 of the fentanyl-positive samples also tested positive for xylazine, according to an article published in April of this year in Morbidity and Mortality Weekly Report. Nationally, xylazine was identified in forensic toxicology samples from 36 of 49 states tested in June 2021

From JAMA article: White House Office of National Drug Control Policy (ONDCP) officially designated fentanyl combined with xylazine as an “emerging threat to the United States,” and last November, the US Food and Drug Administration (FDA) warned clinicians about xylazine's serious risks and provided suggestions on how to manage overdoses in which the drug is suspected. The FDA noted that yohimbine hydrochloride and tolazoline hydrochloride are regularly used by veterinarians to reverse the effects of xylazine in animals, but the drugs aren't known to be safe and effective treatment options for xylazine-involved overdoses in humans. “Xylazine is making the deadliest drug threat our country has ever faced, fentanyl, even deadlier,” Anne Milgram, JD, administrator of the US Drug Enforcement Administration (DEA), said in a March public safety alert.

Furthermore, an increase in xylazine-related overdose deaths was also observed in several other regions across the United States. These include Cook County in Illinois; Jefferson County in Alabama; Milwaukee County in Wisconsin; Harris County in Texas; and the state of New Hampshire. This geographical spread suggests that the use of xylazine is rapidly expanding beyond specific localized areas. People can't tell whether xylazine is in their drug sample just by looking at it. However, BTNX, a Pickering, Ontario, biotechnology company, recently developed rapid test strips to detect xylazine in liquid or powder. The strips sell for $3 apiece in the US—triple the cost of the company's rapid fentanyl test strips—although the distributor's website notes that special pricing is available for nonprofit organizations.

In some places, the situation is so severe that xylazine is appearing in as many as 1 in 5 overdose deaths. This underscores the urgency of addressing this growing public health concern.

### System

The system under discussion pertains to the escalating issue of xylazine use. Xylazine, a substance originally intended for use in veterinary medicine, has seen a rapid increase in misuse among humans. This trend is particularly concerning given the unique properties and effects of xylazine.

Unlike opioids, xylazine acts as a tranquilizer or sedative. This means that it depresses activity in the central nervous system, leading to effects such as drowsiness, relaxation, and a reduction in anxiety. However, these effects can also include dangerous health risks such as respiratory depression, where breathing becomes dangerously slow or even stops, which can lead to overdose deaths.

One of the major challenges in addressing xylazine misuse is that it does not respond to naloxone, also known as Narcan. Naloxone is a medication designed to rapidly reverse opioid overdose by binding to opioid receptors in the brain and blocking the effects of opioid drugs. However, because xylazine is not an opioid and does not bind to opioid receptors, naloxone is ineffective against it.

This presents a significant obstacle in treating xylazine overdoses. Without an effective reversal agent, healthcare providers must rely on supportive care to treat these overdoses. This can include measures such as providing intravenous fluids, assisting with breathing, or treating any other symptoms that arise.

As the use of xylazine continues to rise, experts are striving to understand more about this substance and its risks. Research is ongoing into potential treatment options and strategies to combat this growing public health issue.

### Method

The proposed methods for delivering the antidotes or treatments for a xylazine overdose may include at least one of the following three methods:
a. Inhaled, Intranasal Aerosol Spray: Referring to FIG. 1, disclosed methods involve the use of an aerosol spray mechanism 100 having an aerosol nozzle 110 configured to deliver a spray of fluid from within aerosol spray mechanism 100 out of aerosol nozzle 110 when actuated by a plunger 120. Other intranasal spray-type or liquid delivery devices may be used without departing from the scope of the disclosure. Referring to FIG. 2, a nose 200 of a patient is depicted receiving the medication through a nostril 210 into a nasal cavity 220. The medication may be delivered as an aerosol spray, which allows it to be quickly absorbed through the mucous membranes in the nasal passages. Alternatively, the medication may be delivered into nasal cavity 220 as a regular spray, as a liquid stream, or as drops or droplets. This method is non-invasive and can be self-administered, making it a practical option in emergency situations. Also, the utilization of an aerosol spray may be advantageous in the case where the patient is not breathing or has weakened capacity to breath and therefore is unable to inhale a regular spray or droplets. The aerosolized medication allows the medication to spread over a larger area of intranasal mucous membranes and possibly lung tissue for increased absorption into the bloodstream. Other advantages that exist with the nasal spray is that the patient may be oriented at any angle and that delivery is simple such that no specialized training is necessary such that anyone could deliver the medication including the patient who, although drugged may still have the self-awareness that their drug trip is going badly, and they may be in the process of an overdose situation.
b. Intramuscular Administration by Syringe: Referring now to FIG. 3, an alternative delivery method for a xylazine antidote or for xylazine overdose support medications is depicted. The method involves injecting the xylazine antidote or support medications using a needled syringe 300 directly into a muscle, usually in the upper arm 310, thigh 320, or buttocks 330. Intramuscular injections can be administered by a healthcare professional and allow for the rapid absorption of medication into the bloodstream. However, intramuscular administration may be shied away from by those who have not been trained to do such injections or by the patient. Therefore, intramuscular injection may not be preferred when administration needs to be done in an emergency by an untrained person or by the patient because the administration may not be done correctly, or the administration may not be done at all because the user is afraid to do the injection.
c. Oral, Rapid-Acting Formulation: This method involves the patient swallowing a pill or liquid formulation of the antidote or support medications. Referring to FIG. 4, capsules 400 are representative of a powder containing pill, a gel cap, a liquicap or the like. While oral administration is typically slower than other methods due to the need for the medication to pass through the digestive system, a rapid-acting formulation, or a fast-dissolving sublingual could potentially speed up this process.

Each of these methods has its own advantages and considerations, and the choice of method would depend on various factors such as the patient's condition, the setting in which treatment is being administered, and the specific properties of the antidote.

### Treatment

Although there is no specific antidote to treat humans that overdose on xylazine, such potential treatments are being researched. Hemodialysis has been suggested as a form of treatment but is usually unfavorable due to the large volume of distribution of xylazine throughout the body. Besides supportive therapeutic interventions—treatment includes, but is not limited to, physiological antagonists to central nervous system (CNS) depressants.

Physiological antagonists to CNS depressants are substances that produce effects opposite to those of the depressants. They stimulate the CNS or enhance its activity. For instance, most CNS depressants increase the activity of GABA, an inhibitory neurotransmitter. Therefore, substances that decrease GABA activity or increase the activity of excitatory neurotransmitters may be physiological antagonists to CNS depressants.

Another class of drugs that may be considered physiological antagonists to CNS depressants are sympathomimetic drugs. These drugs mimic the effects of substances produced by the sympathetic nervous system, which is part of the autonomic nervous system that prepares the body for action. Sympathomimetic drugs can increase heart rate, blood pressure, and breathing rate, which are effects opposite to those produced by CNS depressants.

In at least one study, it has been concluded that the effects of xylazine can potentially be reversed by the analeptics 4-aminopyridine, doxapram, and caffeine.

However, the latest research [JAMA article 2] in which Naloxone was administered suggested that bystanders were equipped to respond to overdoses, yet the low proportion of individuals with a pulse on EMS arrival was concerning. Naloxone appeared to be not effective against xylazine, creating a need for swift medical intervention.

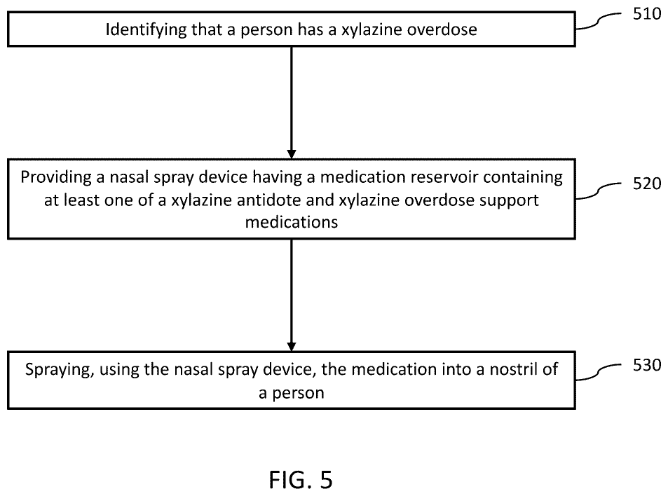

Referring now to FIG. 5 is a flowchart of an example method for treating a xylazine overdose. The method includes at step 510, identifying that a person has a xylazine overdose. At step 520, the method includes providing a nasal spray device having a medication reservoir containing at least one of a xylazine antidote and xylazine overdose support medications. At step 530, the method includes spraying, using the nasal spray device, the medication into a nostril of a person.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g., “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

What is claimed is:

1. A system for treating xylazine introduced into an human body, comprising:
an introducer including a body having an inner volume, a delivery tip, and an actuator; and
medication within the inner volume of the body, the medication including at least one of a xylazine antidote and xylazine overdose support medications;
wherein the introducer is configured to deliver at least a partial dose of the medication, when the actuator is manipulated, through the delivery tip and into a body cavity.

2. The system of claim 1, wherein the actuator includes a flexible bulb.

3. The system of claim 1, wherein the actuator includes a button.

4. The system of claim 1, wherein the introducer is configured to be a nasal introducer and the delivery tip is configured to fit into a nostril of a person.

5. The system of claim 1, wherein the body cavity includes a nasal cavity.

6. The system of claim 1, wherein the delivery tip is configured to at least partially aerosolize the medication.

7. The system of claim 1, wherein the xylazine support medication includes analeptics 4-aminopyridine.

8. The system of claim 1, wherein the xylazine support medication includes doxapram.

9. The system of claim 1, wherein the xylazine support medication includes caffeine.

10. The system of claim 1, wherein the xylazine support medication includes sympathomimetic drugs.

11. A xylazine overdose treatment device, comprising:
an intranasal delivery device, including,
a body including a reservoir configured to hold medication,
a force receiving input device,
a reservoir pressurizing mechanism configured to pressurize the reservoir when the force receiving input device receives a force input, and
an aerosol nozzle coupled to the reservoir and configured to create an aerosol spray when the reservoir is pressurized; and
a medication, disposed in the reservoir, including at least one of a xylazine antidote and xylazine overdose support medications.

12. The device of claim 11, wherein the force receiving input device includes a plunger.

13. The device of claim 11, wherein the force receiving input device includes a flexible bulb.

14. The device of claim 11, wherein the xylazine support medication includes analeptics 4-aminopyridine.

15. The device of claim 11, wherein the xylazine support medication includes doxapram.

16. The device of claim 11, wherein the xylazine support medication includes caffeine.

17. The device of claim 11, wherein the xylazine support medication includes sympathomimetic drugs.

18. A method of treating a xylazine overdose, comprising:
identifying that a person has a xylazine overdose;
providing a nasal spray device having a medication reservoir containing at least one of a xylazine antidote and xylazine overdose support medications; and
spraying, using the nasal spray device, the medication into a nostril of a person.

19. The method of claim 18, wherein the identifying that a person has a xylazine overdose includes dosing the person with naloxone multiple times and observing little improvement.

20. The method of claim 18, wherein the spraying includes an at least partial aerosolized spraying of the medication.
